(19) United States
(12) Patent Application Publication
(10) Pub. No.: US 2008/0311649 A1
(43) Pub. Date: Dec. 18, 2008

(54) Pressurized flexible tubing system for producing Algae

(75) Inventors: George Benjamin Cloud, Chandler, AZ (US); Souren Naradikian, Phoenix, AZ (US); Stephen Irwin, Avondale, AZ (US)

Correspondence Address: ETHERTON LAW GROUP, LLC, 5555 E. VAN BUREN STREET, SUITE 100, PHOENIX, AZ, 85008, US

(73) Assignee: XL Renewables, Inc.

(21) Appl. No.: 12/156,506

(22) Filed: Jun. 2, 2008

Related U.S. Application Data
(60) Provisional application No. 60/932,674, filed on May 31, 2007.

(51) Int. Cl. C12M 1/02; C12M 1/08; C12R 1/89

(52) U.S. Cl. 435/292.1

(57) ABSTRACT

An apparatus for producing algae circulates algae fluid through flexible reactor tubing that is at least partially translucent to sunlight. The reactor tubing lies flat when not pressurized. Preferably, the reactor tubing is made of clear polyethylene with UV inhibitors, the polyethylene being between 6 and 15 mil thick. The reactor tubing preferably has a substantially circular cross-section with a 6 inch diameter and is preferably 1250 feet long. Gas relief valves allow gases generated during algae production to escape from the reactor tubing. CO₂ may be injected into the algae fluid to stimulate photosynthesis. A circulation pump propels the algae fluid through the reactor tubing, keeping the reactor tubing pressurized and stationary without touching the reactor tubing, so that a rigid support structure is not needed. One or more layers of plastic mulch may be disposed above or below the reactor tubing to control temperature and sunlight exposure.

### CROSS-REFERENCE TO RELATED APPLICATIONS

This application claims the benefit of co-pending provisional application No. 60/932,674, filed May 31, 2007.

### FIELD OF INVENTION

This invention relates to algae production systems. This invention relates particularly to a pressurized system of flexible tubing for producing algae.

### BACKGROUND

Microscopic algae are unicellular organisms which produce oxygen by photosynthesis. Microscopic algae, referred to herein as algae, include flagellates, diatoms and blue-green algae; over 100,000 species are known. Algae are used for a wide variety of purposes, including the production of vitamins, pharmaceuticals, and natural dyes; as a source of fatty acids, proteins and other biochemicals in health food products; for biological control of agricultural pests; as soil conditioners and biofertilizers in agriculture; the production of oxygen and removal of nitrogen, phosphorus and toxic substances in sewage treatment; in biodegradation of plastics; as a renewable biomass source for the production of a diesel fuel substitute (biodiesel) and other biofuels such as ethanol, methane gas and hydrogen; to scrub CO₂, NOₓ, VOₓ from gases released during the production of fossil fuel; and as animal feed. With so many uses, it would be desirable to mass produce algae in a low-cost, high-yield manner.

Algae use a photosynthesis process similar to that of higher-developed plants, with certain advantages not found in traditional crops such as rapeseed, wheat or corn. Algae have a high growth rate: it is possible to complete an entire harvest in hours. Further, algae are tolerant to varying environmental conditions, for example, growing in saline waters that are unsuitable for agriculture. Because of this tolerance, algae are responsible for about one-third of the net photosynthetic activity worldwide. Cultivation of algae in a liquid environment instead of dirt allows them better access to resources: water, CO₂, and minerals. It is for this reason that the algae are capable, according to scientists at the National Renewable Energy Laboratory (NREL) (John Sheehan, et al), “of synthesizing 30 times more oil per hectare than the terrestrial plans used for the fabrication of biofuels. The measurement per hectare is used because the important factor in the algae's cultivation is not the volume of the basin where they are grown, but the surface exposed to the sun. Productivity is measured in terms of biomass per day and by surface unit. Thus, comparisons with terrestrial plants are possible. Professor Michael Briggs at the University of New Hampshire estimates that the cultivation of these algae over a surface of 38,500 km², and situated in a zone of high sun-exposure such as the Sonora Desert, would make it possible to replace the totality of petroleum consumed in the United States. Interest in the biotechnology is therefore immense. Arid zones are ideal for the cultivation of algae because sun exposure is optimal while human activity is virtually absent. These algae can be nourished on recycled sources such animal manures. In the context of climatic changes and rising petroleum prices, biofuels are gaining greater acceptance as a renewable energy alternative. Presently, research is being done on microscopic algae that are rich in oils and whose yield per acre is considerably higher than other oilseed crops such as corn and rapeseed. NREL and the Department of Energy are working to produce a commercial-grade fuel from triglyceride-rich micro-algae. NREL has selected 300 species of algae, both fresh water and salt water algae, as varied as the diatoms (general Amphora, Cymbella, Nitzscha, etc.) and green algae (genera Chlorella in particular) for further development.

However, yield can be limited by the limited wavelength range of light energy capable of driving photosynthesis (400-700 nm, which is only about half of the total solar energy). Other factors, such as respiration requirements (during dark periods), efficiency of absorbing sunlight, and other growth conditions can affect photosynthetic efficiencies in algal bioreactors. The net result is an overall photosynthetic efficiency that has been too low for economical large scale production. The need exists for a large scale production system that provides the user a cost effective means of installation, operation and maintenance relative to production yields.

In order to produce optimal yields, algae need to have CO₂ in large quantities in the basins or bioreactors where they grow. Thus, the basins or bioreactors need to be coupled with economical sources of CO₂. Much of the current research and commercial focus is to install bioreactors on-site with thermal power centers and use the flue gas to supply the CO₂. Experimental automated algae cultivation in large basins or bioreactors has taken place in Hawaii, California, and New Mexico to study the effect of basin surface area, pH, and daily and nightly temperature on productivity of these algae. The number and availability of thermal power centers may be somewhat limited, however. An alternate source of CO₂ is produced from organic wastes such as livestock manure and food processing waste. Sources of livestock manure are more plentiful that thermal power centers and, synergistically, the algae produced in an algae bioreactor could be used for the production of fuel and food to be fed to the livestock. More beneficial, in addition to the resultant CO₂ gas, the resultant effluent is a rich source of fertilizer. Therefore, it would be advantageous to locate a bioreactor near diary farm, cattle or pig feedlot, or other such source of CO₂ and fertilizer.

One proposal for a large-scale system uses a series of rigid pipes elevated over an earthen bed. This system suffers some disadvantages, however, because the rigid pipes are expensive to transport and difficult to install and maintain. Another approach developed disclosed in US Pat. Pub. 2007/0048859 uses polyethylene tubes coupled to a rigid roller structure. The flexible bioreactor tubes are made of two layers of 0.01 inch (10 mil) thick polyethylene, and lay between two sets of rigid guard rails. Rollers traverse the tubes to peristaltically move the algae through the bags by pressing down on the top layer of each bag. In one attempt to avoid an outdoor facility, the Japanese government has launched a research program to investigate the development of reactors which would use fiber optic cables (lighting) which would reduce the surface area necessary for their production and ensure better protection against variety contamination. Unfortunately, all these approaches suffer the same significant disadvantage: they require a framework or other structure be built to operate the system. It would be advantageous to avoid having to build a structure or framework, or at least minimize the amount of building required, in order to minimize capital cost, and reduce the difficulty in erecting and maintaining an algae system.

Therefore, it is an object of this invention to provide a large-scale algae production system for algae-based biofuels and animal feed. It is another object to provide an algae production system that has a lower capital cost than elevated rigid piping and other existing systems. It is another object to simplify erection and maintenance of an algae system. It is a further object to locate CO₂ scavenger next to CO₂, NOx, VOx sources, to reduce pollution and minimize transportation. It is another object to locate an animal feed producer next to animals who eat, to reduce pollution and minimize transportation.

### SUMMARY OF THE INVENTION

This invention is a pressurized tubing system for producing algae. The system comprises flexible tubes made of clear, thin-wall extruded polymer. The tubes are laid in parallel rows on a flat earthen bed. Plastic mulch can be laid below and above the tubes for control of temperature, moisture and light exposure. The tubes are connected to a common inlet and outlet line, a circulation pump, control valves, O₂ relief valves and a CO₂ injection system. To grow the algae, an aqueous solution of concentrated algae is injected into the tubing along with sufficient make-up water as necessary to obtain a desired concentration of algae. Simultaneously, CO₂ is injected under pressure into the system. The algae fluid is circulated through the tubing. As it is exposed to sunlight, the algae photosynthesize, consuming CO₂, producing O₂, and reproducing. Once the algae fluid is concentrated enough to harvest, the algae fluid is released through the output valve and the algae are further processed to make biofuels and animal feed, as known in the art.

### BRIEF DESCRIPTION OF THE DRAWINGS

FIG. 1 is a perspective view of a length of reactor tubing under pressure.

FIG. 2 is a perspective view of a length of reactor tubing, not under pressure.

FIG. 3a is a cross-section schematic of the preferred embodiment of a reactor bed.

FIG. 3b is a cross-section schematic of an alternate embodiment of a reactor bed.

FIG. 4 is a cross-section schematic of another embodiment of a reactor bed.

FIG. 5 is a rear view of a tape roller tractor aligned over a reactor bed.

FIG. 6a is a top view schematic illustration of the preferred reactor tubing in a field in circulation mode and another field in harvest mode, with arrows showing algae fluid flow direction.

FIG. 6b is a top view schematic illustration of an alternate reactor tubing in a field in circulation mode and another field in harvest mode, with arrows showing algae fluid flow direction.

FIG. 7a is a top view schematic of one of the fields of FIG. 6a, with arrows showing algae fluid flow direction during the production stage.

FIG. 7b is a top view schematic of one of the fields of FIG. 6b, with arrows showing algae fluid flow direction during the production stage.

FIG. 8 is a top view schematic illustration of one field of the preferred embodiment, with arrows showing algae fluid flow direction.

FIG. 9 is a close-up view of a reactor bed of FIG. 8, with arrows showing algae fluid flow direction.

### DETAILED DESCRIPTION OF THE INVENTION

This invention is a pressurized system of flexible tubing for producing algae. The system uses reactor tubing configured in one or more reactor beds to form a facility of sufficient capacity to meet certain needs when the sunlight is at its most limited, and excess capacity with greater sunlight.

Reactor Tubing

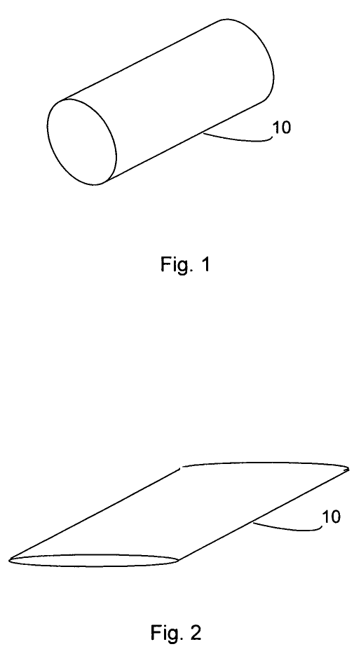

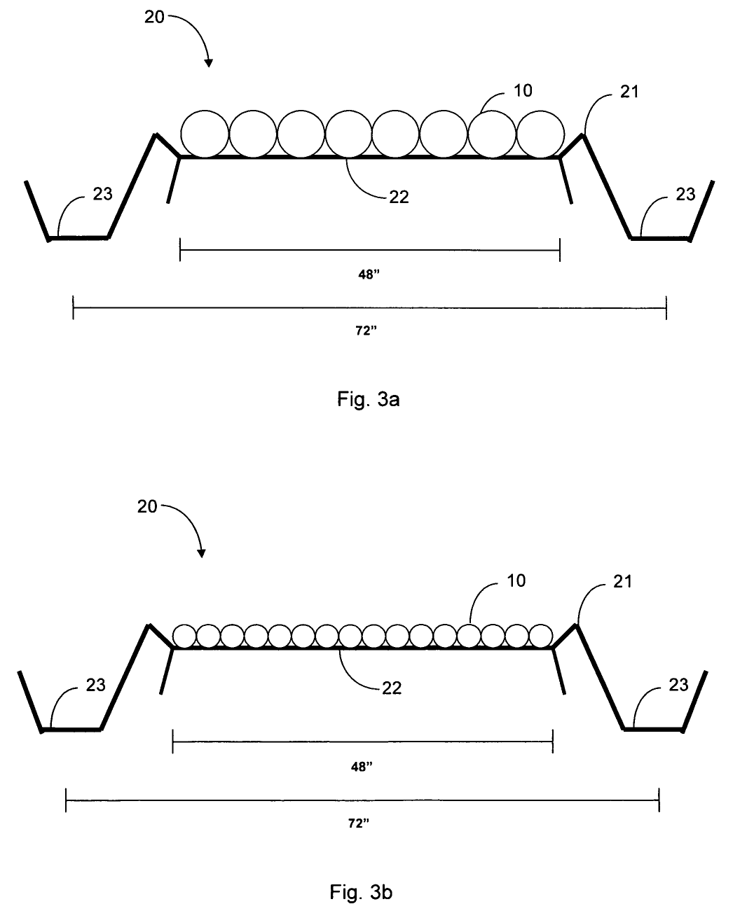

Conventional drip irrigation tubing is made with carbon black, to prevent both UV degradation of the tubing material and algae growth that could cause plugging problems with drip irrigation systems. In contrast, this invention uses tubing without carbon-black so that it has a wall which is at least partially translucent to sunlight, enabling plant material within the tubing to photosynthesize. The modified tubing where the photosynthesis takes place is referred to herein as reactor tubing 10. See FIG. 1. The reactor tubing 10 is also known as tape, because it lays flat when it is not pressurized. See FIG. 2.

The reactor tubing 10 may be made of any flexible, water-impermeable material that can be formed into tubes and will lay flat when not pressurized, particularly any flexible polymer such as phthalated polyvinyl chloride, polyethylene, polypropylene, polyurethane, polycarbonate, and polystyrene. Preferably the reactor tubing 10 is made of clear polyethylene with ultraviolet (UV) inhibitors. The polyethylene may be extruded as one or more sheets which are then bonded together to create the tube, but preferably the polyethylene is extruded as a single tube. This combination of characteristics makes tubing that is very strong, lightweight and durable, yet economical. Using polyethylene reactor tubing is ideal for algae production since the material allows for efficient thermal transfer when the exterior temperature is significantly different than the algae fluid temperature in the tubing. The reactor tubing 10 allows at least some visible light, having 380-750 nm wavelengths, to penetrate the wall and promote algal photosynthesis. Infrared light, having a wavelength of 750 nm-1 mm, and some UV light in the range of 10 nm-380 nm, may also be allowed to penetrate the wall due to their positive effects on photosynthesis, although the UV light may be blocked as explained below.

The reactor tubing 10 may be any shape that permits photosynthesis of the algae traveling through it, including a circle, semicircle, oval, or combination of shapes. Preferably, the reactor tubing 10 has a maximum cross-section of less than 12 inches. More preferably, the reactor tubing 10 is substantially circular with a wall thickness of 10 mil and a 6 inch cross-section. The circular shape allows the reactor tubing 10 to be laid without regard for its orientation, in contrast to a noncircular tube which can only achieve maximum efficacy if oriented along a certain axis. The wall thickness of 10 mil makes the reactor tubing 10 durable and long-lasting while allowing light to penetrate to the bottom of the 6 inch tube except when there is significant accumulation of algae in the reactor tubing 10. The 6 inch diameter is small enough to remain flexible when pressurized, and provides sufficient volume in a single line of reactor tubing 10 so that the turbulence caused by injection of CO2 will circulate the algae from the bottom of the tubing 10 to the top and back down again. Thus, when the algae fluid becomes so concentrated that light cannot penetrate to the bottom of the tubing 10, the stirring action gives all the algae time to photosynthesize. Further, with a 6 inch diameter the tubing 10 may be up to 75% covered by earth, giving the tubing 10 natural insulation from direct sunlight and weather conditions while allowing sufficient sunlight penetration for photosynthesis.

The reactor tubing 10 has a burst rate in excess of 50 psi and an operating pressure of 5 to 20 psi. Over time, the reactor tubing 10 will lose some of its clarity, partially reducing the ability of sunlight to pass through the wall. However, the system design anticipates this potential loss in efficiency by using longer length of reactor tubing 10. While shorter lengths are sufficient to achieve full conversion of the CO₂ to O₂, biofilm accumulation and other variables, such as seasonal temperature variation and degradation of the reactor tubing 10, may negatively affect performance over time. Experimentation determined that a reactor tubing 10 length of 300 feet is considered sufficient to assure optimum system performance in all operating conditions over a five year operating life of the reactor tubing 10.

The life of the reactor tubing 10 is extended dramatically when it is operating under pressure because the wall temperature is less likely to reach temperature levels higher than the contents. Non-use will lower the operating life of the reactor tubing 10 due to wider temperature variations and light intensity. During the summer months, sunlight intensity may cause the polyethylene to degrade rapidly. To extend its life, the reactor tubing 10 may be partially buried as explained above. The reactor tubing 10 may also be made with UV light absorbent molecules. Alternatively, a phosphor or other UV inhibitor, such as a thin layer of common white paint or similar material, can be applied to the reactor tubing 10, making the reactor tubing 10 UV resistant. However, the accumulation of algae in or on the inside of the reactor tubing 10 may also provide sufficient protection against UV intensity.

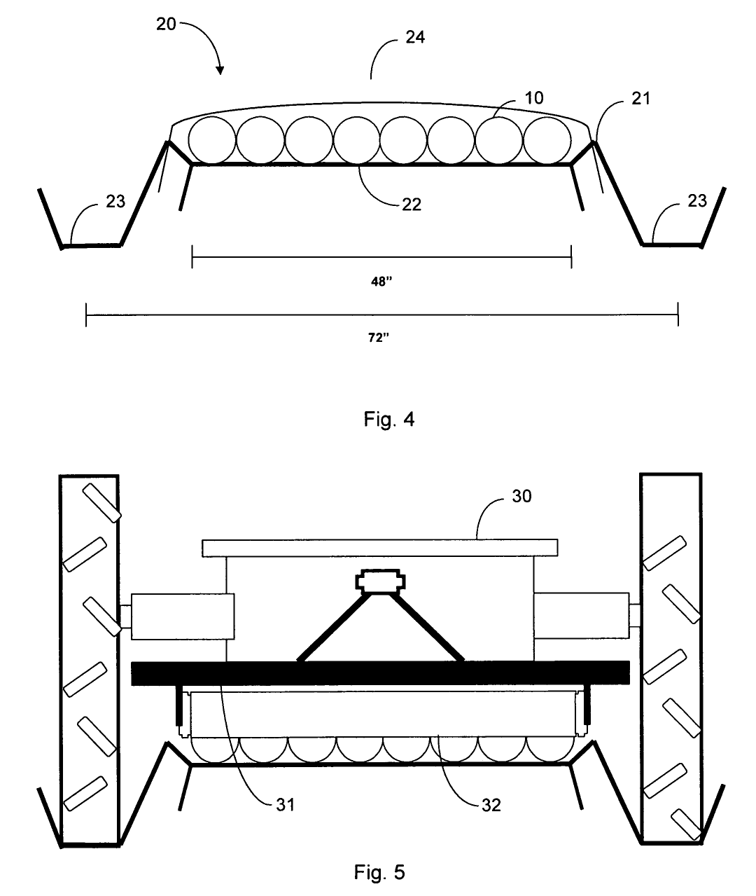

A series of lines of reactor tubing 10 are laid in parallel on top of a raised, earthen bed 21 to create a reactor bed 20. Whether the reactor tubing 10 is pressurized or unpressurized, it will remain in place in the reactor bed 20 without need for a rigid support structure: unpressurized, the reactor tubing 10 lies flat and stationary; pressurized, the reactor tubing 10 will support itself and no support framework is required to create the flow of algae fluid, which is propelled by a pump as described below. To provide the volume of algae fluid per acre that is needed for optimum yields, the preferred embodiment uses a 6 foot wide reactor bed 20 with a series of lines of 6 inch reactor tubing 10 to cover the center 4 foot width, leaving adequate room for drainage in case of rain or system leaks, and paths 23 for equipment to pass over the reactor tubing 10 for system maintenance. See FIGS. 3a and 3b. Each acre of reactor beds 20 requires about 58,080 feet of 6 inch reactor tubing 10 or 116,160 feet of 3 inch reactor tubing 10, as explained in the facility layouts described in more detail below.

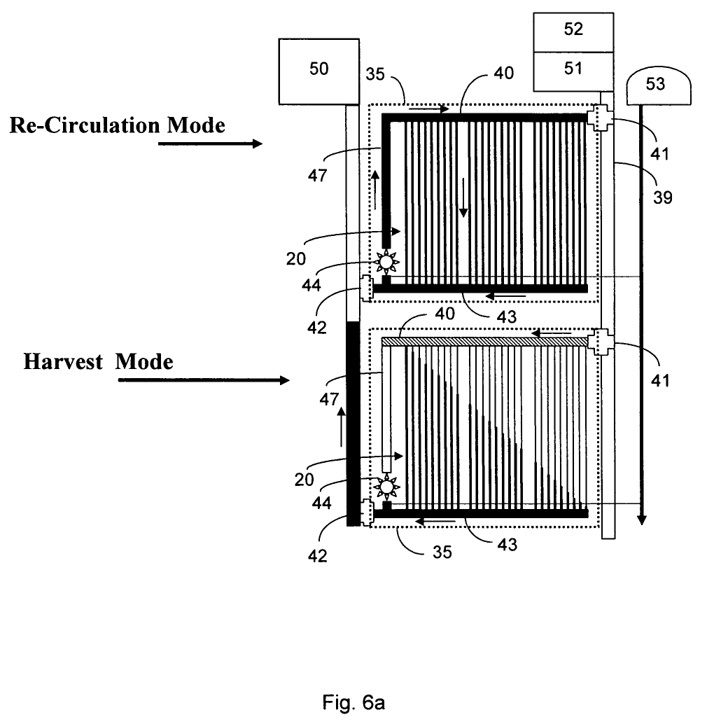

The raised earthen bed 21 is preferably covered with a thermal barrier 22, such as plastic mulch, which serves to maintain the algae fluid temperature and to prevent weed growth that could interfere with production by shading the reactor tubing 10. During winter months, a protective layer 24, preferably of plastic mulch, can be installed that covers the reactor tubing 10. See FIG. 4. The protective layer 24 creates an environment where temperature can be maintained. The parasitic temperature loss of the algae fluid during winter months can be managed by the greenhouse effect where the algae fluid temperature, along with sunlight, would serve to heat the air between the thermal barrier 22 and protective layer 24. The protective layer can be removed seasonally to relieve excess heat during the summer months. Preferably, the edges of the plastic mulch are covered with dirt using mulch laying equipment, such as a mulch-laying tractor. Tractors can straddle each bioreactor bed to travel up and down the rows for periodic maintenance, repair of leaks, and replacement of reactor tubing 10. Alternatively, over-the-row tunnels or miniature greenhouses can be used for temperature control.

Rollers

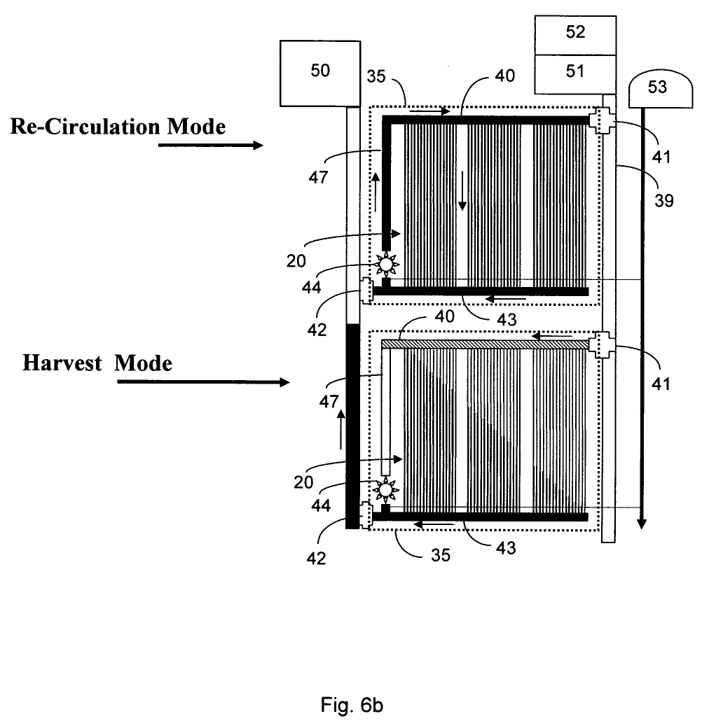

The accumulation of a biofilm or algae attached to the interior wall of the reactor tubing 10 can serve to protect the polyethylene during times of high light intensity. During times of low light intensity in the winter months, however, the biofilm may need to be removed, or its shadowing effect lessened, for optimum production. A number of maintenance options can be used to manage the biofilm buildup. For example, the design length of the bioreactor tube can be longer to accommodate the lower CO₂ conversion rate from a buildup of biofilm. Second, a mechanical agitation method can be used where rollers 32 are mounted on a toolbar 31 pulled through the bioreactor field by a tractor 30. See FIG. 5. The roller 32 applies pressure to the tape 10 and partially collapses the tape 10 as it passes over it, creating a venturi effect that increases the flow rate sufficient to push the algae fluid along the reactor tubing 10 at a high velocity and effectively scour the interior wall of the reactor tubing 10. This is considered the most effective and least costly method of controlling the biofilm accumulation.

Facility and Production

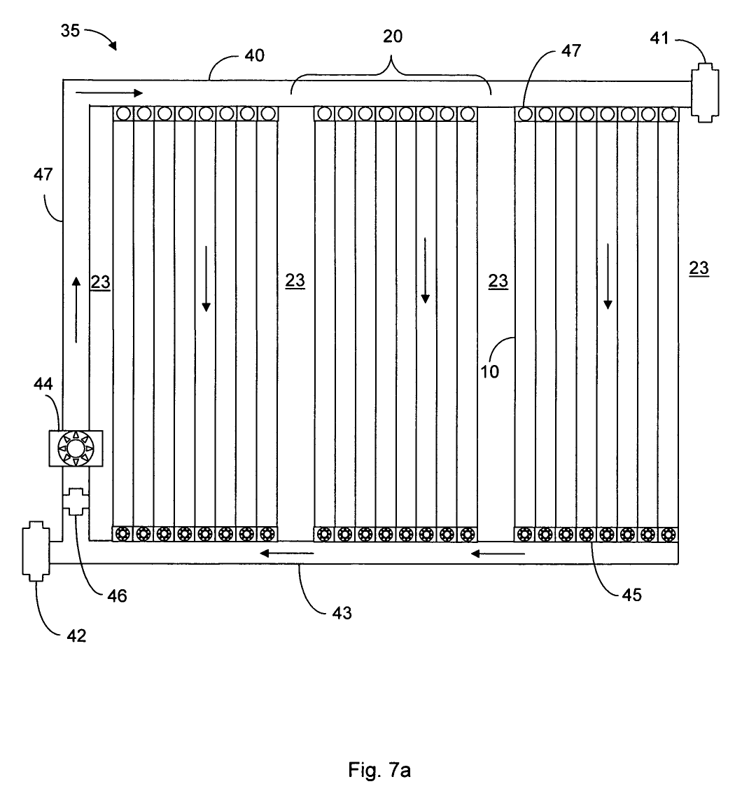

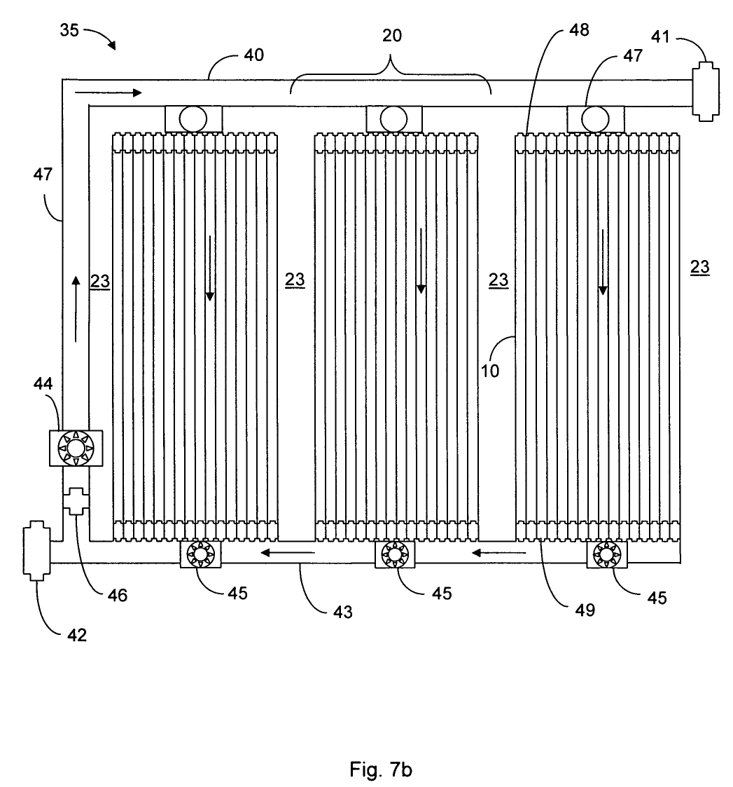

A reactor facility comprises any number of fields 35, each with any number of reactor beds 20. Within each reactor bed 20, reactor tubing 10 can be laid out in any number of configurations, depending on the land available, cost and other factors. FIGS. 6a through 7b illustrate a first embodiment in which the reactor tubing 10 is laid out in a reactor bed 20 in 300 ft lengths. FIGS. 6a and 6b show two fields 35 and FIGS. 7a and 7b are close-up views of the field 35 in recirculation mode of FIGS. 6a and 6b, respectively.

Each field 35 comprises a circulation pump 44, an inlet valve 41, an outlet valve 42, gas relief valves 45, a CO₂ injection system 46, and three reactor beds 20. Using the preferred 6 inch tubing, each reactor bed 20 has 8 lines of reactor tubing 10 connected to the common inlet line 40 with an inlet fitting 47 and connected to the common outlet line 43 with a gas relief valve 45, as shown in FIG. 7a. Using the alternate 3 inch tubing, each reactor bed 20 has 16 lines of reactor tubing 10. Each line of reactor tubing 10 is connected to an inlet manifold 48, which is connected to the common inlet line 40 with an inlet fitting 47, and an outlet manifold 49, which is connected to the common outline line 43 with a gas relief valve 45, as shown in FIG. 7b.

Algae fluid from an algae fluid source 52 is introduced into the facility at the inlet valve 41 in an inlet header line 39. At the inlet valve 41, the algae fluid may be moving slowly or have a substantial velocity, depending on the pressure in the inlet header line 39 and the propulsion means used to transport the algae fluid from the source 52. For example, the source 52 may be a short distance from the field 35, and the algae fluid may be propelled by gravity or by a pump. Preferably, pressure is allowed to build at the inlet valve 41 until the algae flow has enough initial velocity to pass through the reactor tubing 10.

From the inlet valve 41, the algae fluid passes into the reactor tubing 10 which exposes the material to sunlight so that photosynthesis may occur and the algae may reproduce. After the algae fluid travels the length of the reactor tubing 10, the algae concentration increases, as does CO₂ intake and O₂ output. The O₂ and other accumulated gases are released through a gas relief valve 45 near the end of the reactor tubing 10. Each field 35 may have a single gas relief valve 45, or each reactor bed 20 may have a gas relief valve 45 as shown in FIG. 6b, but preferably each line of reactor tubing 10 has its own gas relief valve 45 as shown in FIG. 6a.

During the production stage, the algae fluid then flows through the outlet line 43 towards the circulation pump 44. Prior to passing through the circulation pump 44, CO₂ gas is injected in the outlet line 43 using a CO₂ injector 46. The CO₂ is injected under pressure, which helps it dissolve into the algae fluid stream. Preferably a venture-type injector, such as a Mazzei® injector, is used to inject the CO₂, but other types of injectors may be used. The CO₂ addition under pressure also agitates the algae fluid, thereby keeping the CO₂ in suspension for a higher conversion rate of CO₂ to O₂.

The circulation pump 44 is a stationary cyclic pump, such as a centrifugal, diaphragm, or hydraulic ram pump, that propels the algae fluid through the facility, agitating the algae flow and maintaining adequate pressure in the reactor tubing 10 without touching, pinching, or otherwise causing wear on the reactor tubing 10. In the preferred embodiment, the circulation pump 44 is a large diameter, slow turning, centrifugal pump. The preferred embodiment uses a circulation pump 44 to achieve a maximum flow velocity of about 2 feet per second through the reactor tubing 10. Assuming a reactor bed with 300 feet of reactor tubing 10, it will take at least 2.5 minutes for the algae fluid to pass through the reactor tubing 10 and convert the injected CO₂ to O₂. Pumping the algae fluid under pressure will result in some damage or loss to the growing algae due to the shear force created by the pump 44. This loss is considered to be minimal and not significantly impact the overall production of the system. This risk of yield loss is minimized with the use of a larger size and slower revolution pump 44 to meet the flow rate and pressure rating requirements of the system.

The algae fluid then passes through the recirculation line 47 back into the reactor tubing 10 and the circulation process of loading CO₂ and passing through the reactor tubing 10 repeats until the desired solids content is achieved, generally every few hours depending on light intensity and water temperature, and the algae concentration in the fluid is sufficiently high to harvest the algae. The algae are then harvested by releasing some of the algae fluid through the outlet valve 42 as described below.

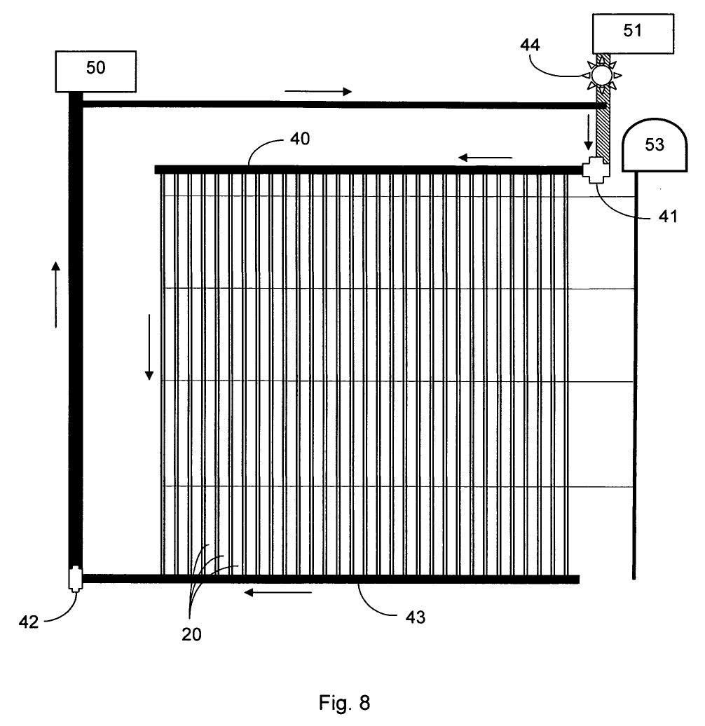

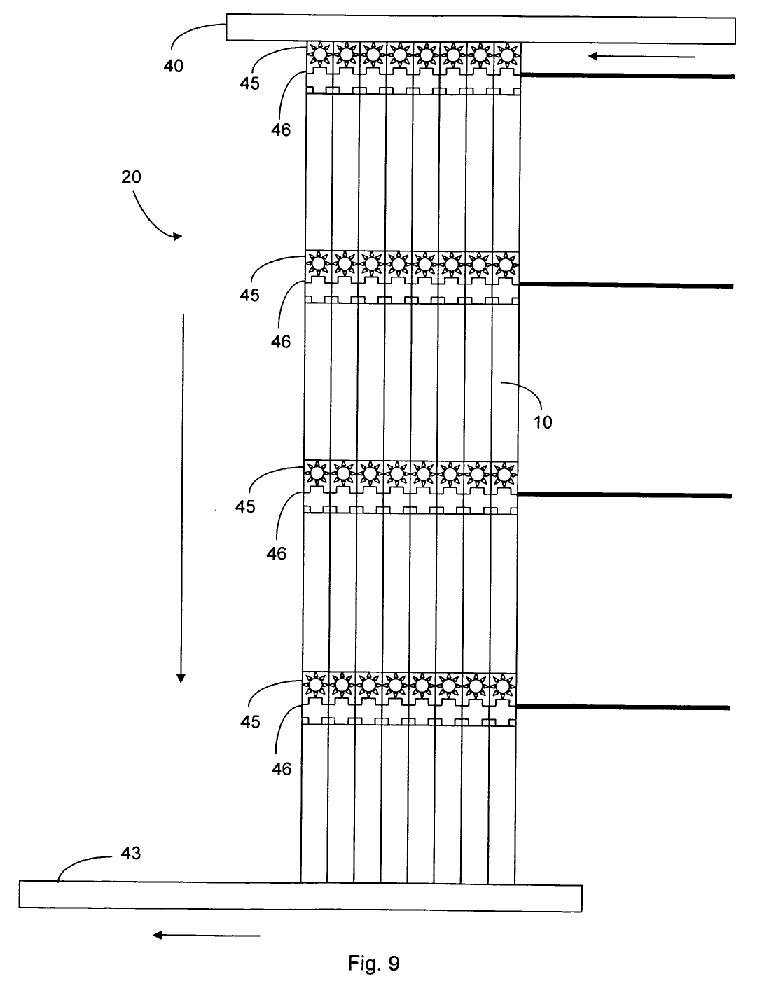

FIGS. 8 and 9 illustrate a second, preferred embodiment. Like the first embodiment, the facility comprises reactor tubing 10 connected to a common inlet line 40 and outlet line 43, a circulation pump 44, an inlet valve 41, an outlet valve 42, gas relief valves 45 and a CO₂ source 53. In this version, however, the preferred 6 inch reactor tubing 10 is 1250 feet long, so that the algae fluid is sufficiently concentrated to harvest in a single pass, making recirculation unnecessary. The alternate 3 inch reactor tubing 10, or another size reactor tubing 10 with a cross section of less than 12 inches, may be used in the preferred embodiment, but only the preferred 6 inch reactor tubing 10 is illustrated in the figures.

FIG. 8 illustrates the facility using a single field 35. Algae fluid is introduced to the inlet line 40 at the inlet valve 41, downstream of the circulation pump 44, which pumps the algae fluid through the system. From the inlet line 40, the algae fluid passes into the reactor tubing 10 which exposes the material to sunlight so that photosynthesis can occur. As the algae fluid travels the length of the reactor tubing 10, the algae concentration increases, as does CO₂ intake and O₂ output. About every 300 feet, accumulated gases are released through a gas relief valve 45 and CO₂ is injected into the reactor tubing 10 with a CO₂ injector 46. The gas relief and CO₂ injection are repeated about every 300 feet, as shown in FIG. 9. The flow rate is designed to pass the algae fluid through the full length of reactor tubing 10 in 3.5 hours. Depending on light intensity and water temperature, and the algae concentration in the fluid when introduced, the outlet flow is sufficiently concentrated to harvest the algae. The algae are then harvested by releasing some of the algae fluid through the outlet valve 42 as described below.

The preferred embodiment comprises the second embodiment as shown in FIG. 8: a single field 35 of 40 gross acres (1320 ft×1320 ft); 33 net acres of reactor beds 20 (1200 ft×1250 ft); 22 net acres of reactor area (792 ft×1250 ft); a flow rate of 700 gpm/field or 3.5 gpm/bed or 0.22 gpm/line; and algae dwell time of 3.5 hours.

The pressurized bioreactor system is scalable. For large scale algae production, a series of reactor fields will be interconnected into a common algae collection point for ease of processing. A reactor field is a series of reactor beds 20 that are supplied by a single inlet valve, circulation pump, CO₂ injection system, air relief valves, and outlet valve. Most of the components may be adapted from common components currently produced and used in drip irrigation systems. These components include low-cost, thin-wall, durable tubing, water and air-relief valves, pvc pipe, mechanical pumps and filters. Each reactor field is designed to provide an adequate dwell time for the algae to convert the injected CO₂ into O₂ through the photosynthesis process by exposing the algae fluid to sunlight.

Harvest Cycle

In the first embodiment, the harvest cycle begins when the algae fluid reaches a desired concentration of algae by simultaneously opening the inlet valve 41 and outlet valve 42 to allow a portion of the concentrated algae fluid to be displaced. When the pre-determined volume of the concentrated algae fluid is disbursed (as usually measured by time), the valves close and the remaining material is blended by the circulation pump 44. Only a portion of the system volume is harvested to lower the solids content to a prescribed level. The displaced algae fluid is delivered to a storage sump 50 where an adequate amount of algae is stored to assure a 24 hour process operation. As shown in FIGS. 6a and 6b, during the harvest cycle the circulation pump 44 and recirculation line 47 are unused as the concentrated algae fluid flows out of the output valve 42, and the input line 40 is flooded with new fertility water (algae fluid diluted in make-up water) from the fertility water source 51. The fertility water flows through the reactor tubing 10, displacing the concentrated algae fluid. Once the valves close, the circulation pump 44 restarts and fertility water is blended with the algae water remaining in the system to start the next production cycle. Thus, the concentration of the algae fluid is diluted during the harvest cycle and then concentrated by algae growth over a sufficient period of time to reach a prescribed concentration level for the next harvest cycle. The algae fluid will be comprised of make-up water and fertility in amounts necessary to optimize algae production and maintain the algae fluid at an ideal range of concentration. The algae content percentage in the water is measured periodically to make sure it is not exceeding a pre-determined limit. Excess algae concentration is easily controlled with the introduction of chlorine or simple dilution.

The preferred embodiment is designed so that the algae fluid reaches a harvestable concentration after a single pass through the reactor tubing 10. At the end of the reactor tubing 10, the algae fluid travels through the outlet line 43 and outlet valve 42. The inlet valve 41 may remain open, introducing new algae fluid into the system as the concentrated algae fluid is displaced. Most of the concentrated algae fluid is deposited in the harvest sump 50 for algae processing. A small amount of algae fluid is diverted back to the circulation pump 44 and mixed with fertility water to be reintroduced into the system at the inlet valve 41. In both embodiments, the harvest cycle is continuous; however the timing, duration, and total volume of each harvest cycle will vary throughout the seasons of the year. The harvest sump 50 may have a filter to create an algae cake for easy harvest and transportation.

Production is affected primarily by the number of daylight hours. To overcome seasonality of the production system and provide a constant supply of biomass for processing 24 hour 7 day per week, the total area of reactor tubing 10 required is determined by the output on the day with shortest daylight hours of the year. As the volume increases with longer daylight hours, a number of lines of reactor tubing 10, usually measured by reactor bed 20, can be idled.

The exposure to the sunlight serves to maintain the operating temperature of the system. Outside temperatures have limited effect on the algae production system since the digester effluent (effluent resulting from solids separation of manure and used as the fertility water) is approximately 100 degrees Fahrenheit and the CO₂ gas may be injected at the same temperature. The use of a heat exchanger (not shown) with the circulation pump 44 may be added if necessary.

After the algae fluid is harvested, it is further processed for its desired use. For example, if used as a biofuel, the first processing step is to macerate the algae to allow for the efficient separation of the oil. Using a centrifuge fat separator, the oil is effectively separated from the water and organic material and the oil deposited into a storage tank ready for processing into biodiesel through any practical means. The remaining organic matter and water is then pumped directly to an ethanol process and begins with the hydrolysis process to convert the carbohydrates to glucose water which is then fermented to make ethanol, CO₂ and stillage. The stillage is then dried to make a high protein animal feed. Any remaining thin stillage can be used to make methane gas in a digester system. The efficiency is created when the algae is produced on-site and can be processed without the need to dry and transport the material.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims.

I claim:

1. An apparatus for producing algae, the apparatus comprising:
a) flexible reactor tubing that has a wall that is at least partially translucent to sunlight; and
b) a stationary circulation pump for moving algae fluid through the reactor tubing.

2. The apparatus of claim 1 wherein the wall is substantially translucent to sunlight.

3. The apparatus of claim 1 wherein the reactor tubing further comprises polyethylene.

4. The apparatus of claim 2 wherein the thickness of the wall is between 6 and 15 mil.

5. The apparatus of claim 1 wherein the reactor tubing further comprises a UV inhibitor.

6. The apparatus of claim 1 wherein the reactor tubing has a substantially circular cross section.

7. The apparatus of claim 6 wherein the cross-section is less than 12 inches when the reactor tubing is pressurized.

8. The apparatus of claim 1 wherein each line of reactor tubing is about 1250 feet long.

9. The apparatus of claim 1 wherein the tubing lies substantially flat when it does not contain a pressurized fluid.

10. The apparatus of claim 1 further comprising a gas relief valve connected to the reactor tubing.

11. The apparatus of claim 1 further comprising a CO₂ injector connected to the reactor tubing.

12. The apparatus of claim 11 wherein the CO₂ injector is a venturi-type injector.

13. The apparatus of claim 1 wherein the circulation pump is centrifugal pump.

14. The apparatus of claim 1 further comprising a layer of plastic mulch disposed above the reactor tubing.

15. An algae production system comprising:
a) a stationary circulation pump;
b) an earthen bed;
c) a thermal barrier laid on top of the earthen bed;
d) a plurality of flexible, substantially translucent polyethylene lines of reactor tubing laid on top of the thermal barrier;
e) an inlet valve connected to the pump and to each line of reactor tubing such that a pressurized fluid may be introduced through the inlet valve into each line of reactor tubing and the fluid flows in the same direction in all lines of reactor tubing; and
f) an outlet valve connected to each line of reactor tubing such that the pressurized fluid may be released from the line of reactor tubing.

16. The algae production system of claim 15 wherein each line of reactor tubing has a maximum cross-section of less than 12 inches.

17. The algae production system of claim 15 wherein each line of reactor tubing is about 1250 feet long.

18. The algae production system of claim 15 further comprising a plurality of gas relief valves attached to the reactor tubing.

19. The algae production system of claim 15 further comprising a plurality of CO₂ injectors attached to the reactor tubing.

20. The algae production system of claim 15 wherein the circulation pump is centrifugal pump.
